(19) United States
(12) Patent Application Publication
(10) Pub. No.: US 2004/0101541 A1
(43) Pub. Date: May 27, 2004

(54) Electrolytic cream

(76) Inventors: Michael Scully Heffernan, Fulton, TX (US); Jennifer Anne George, Fulton, TX (US)

Correspondence Address: Dr. Michael Heffernan, PhD, P.O. Box 2321, Fulton, TX, 78358, US

(21) Appl. No.: 10/304,168

(22) Filed: Nov. 27, 2002

(51) Int. Cl. A61K 7/00; A61K 33/38; A61K 33/32; A61K 33/24

(52) U.S. Cl. 424/401; 424/618; 424/642; 424/649

(57) ABSTRACT

In the past topical creams and lotions were mainly designed to be used to reduce pain and inflammation, and not necessarily to promote healing or regeneration. When existing remedies were designed to promote healing they were designed to do so by chemical mediation, e.g. by removing pain and/or blocking inflammatory produced substances produced in reaction to injury. The current invention is designed to operate entirely by a different mechanism of electrolytic induction. Using a composition of atomic metals suspended in a water based emulsion and suspension negative ion and polarity is induced in areas of the body where needed.

### BACKGROUND AND SUMMARY

Subject matter of invention pertains to healing. Chronic pain and injury in non healing joints and soft tissues of the human body claim substantial costs in lost productivity, and contribute to considerable healthcare expenditure. Current products focus on pain relief, but few if any are designed to promote healing and regeneration. Prior research by Dr. Robert Becker identified negative electric charges as vital to regeneration and healing. The current invention incorporates this finding by establishing an small electrolytic charge and current at and near an injury site using a cream containing a combination of metals in micronized form applied to the body surface over points of tissue degeneration and pain.

### DESCRIPTION

The electrolytic cream consists of micronized (5-10 micron sized) zinc, gold, and silver, suspended in an aqueous base of aloe vera gel. The composition is created by mixing 100 grams of zinc, 10 grams of gold and 20 grams of silver in 1 liter of the gel. This mixture is then applied over arthritic, or soft tissue damaged joints, ligaments, tendons, and/or muscles once per day for a periods of six weeks. This electrolytic cream is unique from other inventions because it uses a combination of metals in their atomic state to induce a negative charge or polarity over injured and chronically damage tissue. Other creams do not do this nor are they intended to due this. For example silver is used topically as a antibacterial, but it is used in molecular or combined form, usually silver chloride, and therefore in this form has no electrolytic properties.

1. What we claim as our invention is all topical creams, salves, and lotions with a composition that is designed to produce an electrolytic negative polarity over damaged or painful tissue for purposes of healing, pain relieving, or regenerative purposes. The cream is to be used on all mammalian species, especially humans.
